(19) United States
(12) Patent Application Publication
(10) Pub. No.: US 2008/0213414 A1
(43) Pub. Date: Sep. 4, 2008

(54) Compositions containing phaseolus vulgaris extract and alpinia officinarum extract for the prevention and treatment of obesity and type II diabetes

(76) Inventor: Di Pierro Francesco, Trezzano sul Naviglio (IT)

Correspondence Address: Walter H. Schneider, 21530 Beechwood Rd, Circleville, OH, 43113, US

(21) Appl. No.: 12/077,892

(22) Filed: Mar. 21, 2008

Related U.S. Application Data
(62) Division of application No. 11/321,317, filed on Dec. 29, 2005, now abandoned.

(51) Int. Cl. A61K 36/9062; A61P 3/10; A61P 3/04

(52) U.S. Cl. 424/773

(57) ABSTRACT

A process for treating an individual suffering from obesity and type II diabetes in which said individual is caused to ingest a composition comprising (1) a kidney bean extract with a standarized phaseolamine content and (2) Alpinia officinarum extract with a standardized 3-methylethergalangin.

This is a divisional application of U.S. application Ser. No. 11/321,317 filed Dec. 29, 2005.

### FIELD OF INVENTION

This invention relates to compositions containing Phaseolus vulgaris extract and Alpinia officinarum extract for the prevention and treatment of obesity and type II diabetes.

### PRIOR ART

The typical diet of the socio-economically developed countries leads to a significant increase in the incidence of disorders associated with overweight, obesity and type II diabetes.

Their common denominator, namely an excessive rise in the blood glucose curve, explains many of the consequences of these disorders.

Drastic changes in diet alone would obviously lead to a significant improvement in the situation, but drugs which can assist and support the desirable (but not always possible) dietary changes are still useful.

Eating complex carbohydrates (pasta, bread, rice, potatoes, etc.) is known to generate the production of pancreatic alpha-amylase, which is responsible for the digestion of glucose polymer starches consisting of 3 to 9 sub-units. Intestinal maltase and alpha-dextrinase cause the digestion of these short polymers with free glucose units, which generates the blood glucose curve. An increase in blood glucose produces the “insulin peak”, which literally cleanses the blood of free glucose by sending it to the brain and storing it in the muscles and liver in the form of glycogen rosettes. As the muscles and liver are usually already full of these rosettes, the action of insulin leads to the transformation of free glucose into fat deposits (adipose tissue).

Alpha-amylase inhibitors able to prevent this cascade of events are known. Phaseolamin, a heat- and gastro-unstable protein of approximately 55 KD, obtained by extraction with hot water from the fruit of Phaseolus vulgaris (the kidney bean), is well known to be a powerful alpha-amylase inhibitor at very low molarities. Phaseolamin is available on the market with various degrees of specific activity, depending on the commercial source, and is present in various diet supplements and dietetic products.

As phaseolamin is a protein, it is broken down and consequently deactivated by the gastric juices. Gastroprotection of phaseolamin allows over 98% to be released into the enteric environment, and allows the inhibition of pancreatic alpha-amylase and blocking of the cascade of events that normally leads to a rise in the blood glucose curve.

Italian patent application no. MI2004A000313, filed by the Applicant, discloses the fact that gastroprotected phaseolamin is more active and allows a reduction in daily dose or the use of a phaseolamin whose specific activity is not particularly high. According to that patent application, gastroprotection is obtained with a coating of shellac (or another compatible polymer which is able to perform the same function and approved for nutritional use for regulatory purposes) which enables 98% of the enzyme to be released in active form into the enteric environment, thus allowing inhibition of pancreatic alpha-amylase (released in the stomach) and preventing the cascade of events that normally leads to a rise in the blood glucose curve.

Alpinia officinarum, also known as lesser galangal, is a plant belonging to the Zingiberaceae family originating from China, where it is used in traditional medicine as a digestive, antiemetic, carminative, antibacterial and anti-inflammatory agent. The active constituents present in the rhizome include galangol, galangin, prostaglandins, benzoic and oxalic cineolacids, starches and kaempferol.

The inhibiting action of a particular fraction of Alpinia officinarum extract on pancreatic lipase was recently demonstrated. In particular, the fraction soluble in ethyl acetate, enriched and with a standardised content of 3-methylethergalangin, the component responsible for inhibition of pancreatic lipase, with an IC₅₀ of around 1 mg/ml and consequently at a highly acceptable molarity, has been identified. This inhibition reduces the absorption of lipids and triglycerides. The plasma evaluations indicate the high statistical significance of this direct lipid-reducing effect on the triglycerides. However, this fraction, and the resulting lipase inhibition, does not produce a cholesterol-lowering effect, thus providing a further demonstration that it only has a direct, specific action on pancreatic lipase.

3-methylethergalangin, or an enriched fraction thereof, does not require gastroprotection, as the compound is not broken down by the gastric juices.

### DESCRIPTION OF THE INVENTION

It has now been discovered that the association of Phaseolus vulgaris extract with a standardised phaseolamin content and Alpinia officinarum extract with a standardised 3-methylethergalangin content is particularly effective for the prevention and treatment of obesity and type II diabetes.

This invention consequently relates to compositions containing Phaseolus vulgaris extract with a standardised phaseolamin content and Alpinia officinarum extract with a standardised 3-methylethergalangin content for the prevention and treatment of obesity and type II diabetes.

More particularly, the compositions according to the invention contain phaseolamin and 3-methylethergalangin in the ratio of 1:5.

The compositions to which this invention relates are in gastroprotected form, to prevent the breakdown of phaseolamin on contact with the gastric juices and to guarantee the stability of 3-methylethergalangin even at a pH of 1.

According to a preferred aspect, the compositions according to the invention will contain Alpinia officinarum extract in ethyl acetate, with a standardised 3-methylethergalangin content.

According to a preferred aspect, the compositions according to the invention will contain Indena phaseolamin standardised from 5 to 18% (with a phytohaemagglutinin content of between 0.01 and 0.06%). This phaseolamin will be gastroprotected according to the process described in Italian patent application no. MI2004A000313.

The phaseolamin content of the compositions according to the invention will range between approx. 0.1 and approx. 1000 mg, preferably between 2 and 10 mg.

The 3-methylgalangin content of the compositions according to the invention will range between approx. 0.1 and approx. 500 mg, preferably between 1 and 100 mg.

The compositions according to the invention cause a reduction in the blood glucose peak and the postprandial lipid peak greater than that generated by the sum of the effects obtained after separate administration of the individual constituents of the association, apparently due to synergy between the individual constituents.

The compositions according to the invention will preferably be taken a few minutes before meals, to ensure that the product arrives when pancreatic secretion has begun and just before emptying of the stomach, with arrival of the food at the same level. This administration will reduce the absorption of free sugar, lipids and triglycerides, with a consequent calorie reduction and a reduced risk of obesity and diabetes.

The compositions according to the invention could be formulated suitably for oral administration, and will be prepared according to conventional methods well known in pharmaceutical technology, such as those described in Remington's Pharmaceutical Handbook, Mack Publishing Co., N.Y., USA, using excipients, diluents, fillers and anti-caking agents acceptable for their final use.

Examples of formulations according to the invention are set out below.

### EXAMPLE 1

### Gastroprotected Tablets

| INGREDIENT | mg | % |
|---|---|---|
| Alpinia galanga | 50.000 | 12.500 |
| Concentrated kidney bean protein | 30.000 | 7.500 |
| Dicalcium phosphate | 139.987 | 34.997 |
| Microcrystalline cellulose | 121.470 | 30.368 |
| Shellac | 15.000 | 3.750 |
| Croscarmellose sodium | 12.000 | 3.000 |
| Hydroxypropyl methylcellulose | 8.450 | 2.113 |
| Talc | 7.713 | 1.928 |
| E171 colouring | 2.831 | 0.708 |
| Triethyl citrate | 1.974 | 0.493 |
| Stearic acid | 1.300 | 0.325 |
| Ammonium carbonate | 1.012 | 0.253 |
| Vegetable magnesium stearate | 4.000 | 1.000 |
| Silicon dioxide | 4.000 | 1.000 |
| Yellow iron oxide | 0.263 | 0.066 |
| TOTAL | 400.00 | |

### EXAMPLE 2

### Mouth-Soluble Sachets

| INGREDIENT | mg | % |
|---|---|---|
| Alpinia galanga | 50.000 | 2.77778 |
| Concentrated kidney bean protein | 30.000 | 1.66667 |
| Mono- and diglycerides of fatty acids | 100.000 | 5.55556 |
| Fructose | 528.000 | 29.3333 |
| Sorbitol | 524.000 | 29.1111 |
| Fruit oligosaccharides | 500.000 | 27.7778 |
| Flavouring | 50.000 | 2.77778 |
| Silicon dioxide | 15.000 | 0.83333 |
| Anhydrous citric acid | 3.000 | 0.16667 |
| TOTAL | 1800.00 | |

### EXAMPLE 3

### 0-Shaped Capsules

| INGREDIENT | mg | % |
|---|---|---|
| Alpinia galanga | 50.000 | 12.500 |
| Concentrated kidney bean protein | 30.000 | 7.500 |
| Mono- and diglycerides of fatty acids | 100.000 | 25.000 |
| Microcrystalline cellulose | 58.000 | 14.500 |
| Dicalcium phosphate | 59.000 | 14.750 |
| Silicon dioxide | 4.000 | 1.000 |
| Magnesium stearate | 4.000 | 1.000 |
| Gelatin shell | 95 | |
| TOTAL | 400.000 | |

1. A process for treating an individual suffering from obesity and type II diabetes in which said individual is caused to ingest a composition comprising (1) a kidney bean extract with a standarized phaseolamine content and (2) Alpinia officinarum extract with a standardized 3-methylethergalangin content.

2. A process according to claim 1 in which the phaseolamin and 3-methylethergalangantin are in a ratio of 1:5 and gastro-protected.

3. A process acording to claim 2 containing phaseolamin standardized to 18% with a phytohaemagglutinin content of 0.06%.

4. A process according to claim 3 in which the composition contains Aplinia officinarum as an extract in ethyl acetate, with a standardized 3-methyethergalangin content.
